(19) United States
(12) Patent Application Publication
(10) Pub. No.: US 2005/0089535 A1
(43) Pub. Date: Apr. 28, 2005

(54) Delivery of biologically active agents

(76) Inventors: Rodney William Kelly, Edinburgh (GB); Andrew Alexander Calder, Edinburgh (GB)

Correspondence Address: Edwin V Merkel;Nixon Peabody, Clinton Square, PO Box 31051, Rochester, NY, 14603, US

(21) Appl. No.: 10/470,984

(22) PCT Filed: Feb. 11, 2002
(86) PCT No.: PCT/GB02/00557

(51) Int. Cl. A61K 9/00

(52) U.S. Cl. 424/400

(57) ABSTRACT

A method of delivering a biologically active agent to the cervix, the method comprising using a needleless injector. Typically, the biologically active agent is a cervical ripening agent. The needleless injector may be a powder injector or a liquid injector.

The present invention relates to the delivery of biologically active agents to the female cervix and, in particular, it relates to the delivery of cervical ripening agents.

The cervix acts as a barrier to the ingress of infection as well as a rigid barrier at the neck of the womb. This organ has relatively little muscle with the majority of the strength of the organ derived from collagen. In a normal, easy delivery, the cervix has to soften and stretch (eface) to allow the baby's head to pass through, if this can be achieved with little uterine contractility then subsequent contractions can deliver the baby with minimal trauma for mother and child.

Biologically active agents such as prostaglandins and chemokines are delivered to the cervix or vaginal fomix for various purposes. In particular, agents are delivered to aid cervical ripening for a number of reasons including (a) the induction of labour, (b) to soften an unfavourable cervix during labour, (c) to assist medical termination of pregnancy, (d) to assist uterine surgery and (e) to accelerate normal parition and to reduce the accompanying risks and discomforts. U.S. Pat. No. 5,624,670 to Kelly et al describes the use of interleukin-8 (IL-8) for inducing cervical ripening, and U.S. Pat. No. 5,908,829 to Kelly describes the use of monocyte chemotactic peptide-1 (MCP-1) for inducing ripening of the cervix.

Vasodilators or their precursors (such as the nitric oxide donor nitroglycerine) are also delivered to the cervix.

At present, biological agents are delivered to the cervix or vaginal fornix using gels, creams, pessaries. A relatively large amount of drug is currently needed since diffusion through the epithelial layers of the cervix is relatively slow. The gel can also get displaced. The most common current method of cervical ripening is to use prostaglandin E₂. This is used as a vaginal gel or tablet or as a gel placed in the cervix. Typically, gel is placed in the vaginal fornix. The prostaglandin has to diffuse through the epithelium of the cervix and act on the blood vessels to increase permeability. In addition prostaglandin may act on fibroblasts to release interleukin-8 (IL-8) within the cervix. The combination of IL-8 and PGE is very powerful in attracting neutrophils (Colditz (1990) Immunol. Cell Biol. 68, 397-403) and neutrophils are a source of collagenase.

There are several disadvantages to the current approaches. Firstly, in order that sufficient amounts of the biologically active agent are delivered to the site of action, relatively high concentrations of the agent need to be included in the formulation administered. Secondly, prostaglandin gels used to ripen the cervix by administration to the vaginal fornix can stimulate the myometrium to contract excessively and, in extreme circumstances, an uterine rupture can occur (Maymon et al (1991) Am. J. Obstet, Gynecol. 165, 368-370). Thirdly, with prostaglandin gel the rate of diffusion into the cervix is limited and variable. Fourthly, for certain of the biologically active agents delivered to the cervix or vaginal fornix, diffusion is relatively slow due to the increased molecular size and hydrophilic nature of the compound. This is particularly true for peptide agents.

During its ripening, the cervix is thought to soften from the internal os. That is likely because of the possible fetal contribution to the timing of birth. Thus, the initial softening might involve agents (such as PGE₂ and or chemokines) diffusing outward from the fetal membranes (amnion and chorion) to the point of contact with the cervix, the internal os. Attempts to deliver chemokines has identified a problem with the diffusion of the active agent, particularly when that agent is a peptide and accordingly may not be lipophilic.

In addition, we believe that delivery to the cervix of any ripening agent should be diffuse rather than punctate and thus conventional injection using a needle and syringe is not ideal.

There remains the need to improve on delivery methods to the cervix and, as described in more detail below, the inventors believe that alternatives lo which use needleless injectors may lead to benefits over the existing methods.

Needleless injectors are well known in the art and have been used to deliver agents principally to the skin of individuals, for example in vaccinations. As far as the inventors are aware, despite needleless injectors having been available for very many years, no-one has suggested that they would be useful in delivering agents to the cervix, and no-one has realised the potential benefits in terms of the possibility of using lower concentrations of biologically-active agents, particularly cervical-ripening agents such as prostaglandins, and benefits in terms of potentially simplifying the formulation of the active agents for delivery. In particular, compared to other delivery methods, no gel matrix (such as a methyl cellulose or modified starch gel) is required.

Commercially available needleless injectors fall into two main categories: those which use powder formulations of the biologically active agent, and those that use the agent in a solution formulation. These will be referred to as “powder” injectors and “liquid” injectors. Generally, both types of injector use compressed air, or gas, or some type of mechanical configuration to “inject” the active agent into the patient.

Powderject (Oxford, UK) manufactures commercially powder injectors. Typically, powder injectors make use of dry powder formulations and fine particles of the drug to be delivered are projected at high velocity within a brief burst of, typically, helium gas. Transdermal application has been described and, typically, the dose is delivered over a skin area of up to 2 cm².

WO 94/24263 (Oxford Biosciences Limited) describes a needleless syringe which uses supersonic gas flow for particle delivery. The needleless syringe has a membrane which is ruptured by gas pressure to generate a supersonic gas flow in which particles containing a therapeutic agent are injected. WO 96/12513 (Oxford Biosciences Limited) describes a similar system except that a soft-walled capsule, such as one with a gelatine wall, is used to hold the therapeutic agent in the syringe. WO 96/20022 (Oxford Biosciences Limited) again describes a similar device except that it has a diaphragm which snaps from inverted to everted positions to catapult particles outwardly.

WO 96/25190 (Oxford Biosciences Limited) describes a needleless powder injector with a bend.

WO 97/34652 (Powderject Research Limited) relates to a shroud which surrounds the nozzle of the syringe and reduces the noise.

WO 98/13470 (Powderject Vaccines, Inc) relates to a rotational flow element which modifies gas flow through the device.

WO 99/01168 and WO 99/01169 (Powderject Research Limited) relate to a prefilled capsule for use with a powder injector and a modified gas release mechanism, respectively.

WO 00/54827 (Powderject Research Limited) relates to a diaphragm for use with a needleless injector wherein the diaphragm provides for enhanced particle retention.

WO 00/62846 (Powderject Research Limited) relates to a needleless powder injector device that does not require particles to be between burstable membranes.

Certain other patent applications relate to the manufacture of particle compositions (WO 97/48485 and WO 00/53160; Powderject Research Limited); and cartridges for use in a needleless powder injector (WO 98/21364; Powderject Vaccines, Inc and U.S. Pat. No. 5,780,100 to McCabe and Heinzen).

Weston Medical (Peterborough, UK) manufactures commercially liquid injectors.

WO 93/03799 (Weston) and WO 95/03844 (Weston Medical) describe a liquid injector system which is used to deliver vaccines by penetration of the epidermis for intradermal delivery. WO 96/28202 (Weston Medical) and WO 97/37705 (Weston Medical) describe a spring-powered dispensing device which can be in the form of a needleless injector. EP 0 520 571 A (Glaxo) describes an atomising nozzle for use with a liquid injector. WO 96/15821 (Weston Medical) describes a glass container for use in needleless injectors, and WO 97/22375 (Weston Medical) and WO 97/36785 (Weston Medical) describe a needleless injector drug capsule and filling method.

WO 96/19252 (Weston Medical) describes a filling device for a needleless injector. WO 00/10630 (Weston Medical) describes an actuator for a gas powered needleless injector. WO 00/15281 (Weston Medical) describes a needleless injector cartridge.

A first aspect of the invention provides a method of delivering a biologically active agent to the cervix, the method comprising using a needleless injector.

The delivery may be directly to the cervix or may be via the vaginal fornix, which is a fold in the vagina where the cervix is located. Thus, the end of the needleless injector may occupy the vaginal fornix and delivery may be at or through the vaginal fornix into the cervix.

Typically, injections will aim at the cervix but may be aimed from different angles.

Preferably, the site of injection is “off-centre” into the cervical lumen or into any part of the externally presenting area of the cervix.

The needleless injector may be any suitable man-made needleless injector and, conveniently, may be a liquid injector or a powder injector as described above. Thus, suitable needleless liquid injectors include those manufactured by Weston Medical Limited, Peterborough, UK and those described in WO 93/03779, WO 95/03844, WO 96/28202, WO 97/37705 and WO 00/10630, all of which are incorporated herein by reference.

Suitable needleless powder injectors include those manufactured by Powderject Research Limited, Oxford, UK and those described in WO 94/24263, WO 96/12513, WO 96/20022, WO 96/25190, WO 97/34652, WO 98/13470, WO 99/01168, WO 99/01169, WO 00/54827 and WO 00/62846, all of which are incorporated herein by reference.

The biologically active agent may be loaded into the injector by means known in the art.

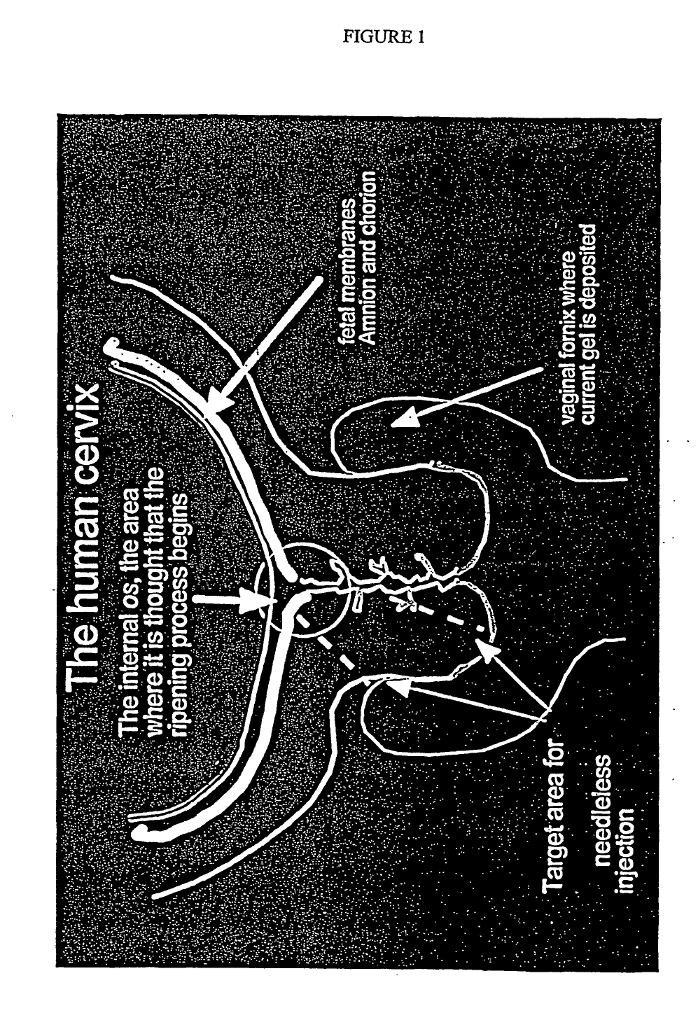

It may be useful if the needleless injector has a “bent” configuration such that delivery of the biologically active agent is to the cervix rather than to the walls of the vagina or to the entrance of the cervix. Thus, the needleless injector may be adapted for this purpose, and the geometry of the device arranged such that there is easy passage through the vagina but that the exit from the injector (ie outlet orifice) is angled such that the biologically active agent is delivered efficiently to the desired site on the cervix. The type of injectors described in WO 96/25190 may be particularly suitable; however, rather than the internal angle of approximately 90° shown in FIGS. 1 to 4 of WO 96/25190, it may be more appropriate if the internal angle of the head to the shaft is between 20° to 90°; preferably between 35° and 85°, for example 65°.

The arrangement of the head containing the outlet orifice shown in FIG. 16B of WO 00/54827 may also be particularly suited for use in the present invention. Similarly, the arrangement shown in FIG. 5 of WO 00/62846, where the exit plane is not perpendicular to the longitudinal axis of the nozzle, but angled, may be particularly suited for use with the invention.

The biologically active agent may be any agent for delivery to the cervix which has a useful effect. For example, the agent may be any agent which has a therapeutic or prophylactic effect, or which is useful in any medical procedure. Typically, the agent is one which is useful in gynaecology or obstetrics.

Suitable agents include prostaglandins (both natural and synthetic), vasodilators or their precursors (such as the nitric oxide (NO) donor, nitroglycerine), and chemokines or any cytokine that could stimulate monocyte or granulocyte entry into the cervical tissue such as GM-CSF.

It is particularly preferred if the agent is a cervical ripening agent, such as a prostaglandin (in particular, prostaglandin E₂) or interleukin-8 (IL-8) or monocyte chemotactic pepfide-1 (MCP-1) or combinations thereof. MCP-1, IL-8 and variants thereof may be obtained by chemical synthetic methods or by recombinant DNA means as is well known in the art. Prostaglandins, such as PGE₂, are commercially available, for example from Pharmacia and Upjohn. The use of prostaglandins in cervical ripening is well known in the art. The use of IL-8 and MCP-1 in cervical ripening is described in U.S. Pat. Nos. 5,624,670 and 5,908,829, respectively, incorporated herein by reference. Variants of MCP-1 or IL-8 which are able to induce cervical ripening may also be used.

A “variant” refers to a protein wherein at one or more positions there have been amino acid insertions, deletions, or substitutions, either conservative or non-conservative, provided that such changes result in a protein whose basic properties, for example cervical ripening-inducing activity, thermostability, activity in a certain pH-range (pH-stability) have not significantly been changed. “Significantly” in this context means that one skilled in the art would say that the properties of the variant may still be different but would not be unobvious over the ones of the original protein.

By “conservative substitutions” is intended combinations such as Gly, Ala; Val, Ile, Leu; Asp, Glu; Asn, Gln; Ser, Thr; Lys, Arg; and Phe, Tyr.

Such variants may be made using the methods of protein engineering and site-directed mutagenesis as described below.

The dose of the agent administered is any suitable dose of the agent which provides the desired effect of the agent. Typically, because of the precision with which the agent can be administered with the needleless injector, the dose may be significantly reduced compared to the dose administered by other, conventional means. The amount may be reduced to 50% or 20% or 10% or 5% or 2% of the amount normally used, depending on the agent administered.

For example, 2 to 100 μg of IL-8 or MCP-1 dissolved in 200 μl of sterile saline may be used in a liquid injector.

Diffused delivery is achieved using needleless injectors. The agent, once injected, distributes radially and along the length of the injection path. Such a method of delivery is not obtainable by any other method of injection, and, although not being bound by any theory, is believed to be particularly beneficial in the context of the present invention.

Administration of lower amounts of the agent may reduce the chance of side effects. For example, side effects of NO donors are headaches and excessive bleeding. Injection of a low amount directly into the cervix would both accelerate the effect and reduce the side effects.

Typically, the needleless injector is loaded with the biologically active agent which is to be delivered to the cervix, the injector is inserted into the vagina such that the exit from the injector (ie outlet orifice) is in proximity to the cervix, and the injector is actuated so as to deliver the agent to the cervix. Preferably, the exit from the injector (ie outlet orifice) is not at the rim of the cervix but is at the side of the cervix.

It will be appreciated that position or type of “head” of the needleless injector (ie that contains the outlet orifice) may be adjusted or adapted depending on the particular use. Thus, particular positions or designs of head may be more suited to delivery when the cervix is partially dilated (eg during parturition) or may be more suited to delivery when the cervix is closed or substantially closed.

A description of the anatomy of the vagina, cervix and uterus is given in FIG. 1 and positions for possible delivery to the cervix are indicated.

The method may be used to deliver biologically active agents to any female mammal, but it is particularly preferred if the method is used to deliver agents to a human female. However, the method may be especially useful for sheep where in vitro fertilization and embryo transfer procedures currently involve laparoscopy which brings up ethical and animal husbandry considerations, the problem being that the cervix is impenetrable. It is believed that a diffuse injection of cervical ripening agent would give a very rapid response, allowing a wider use of in vitro fertilization.

It will be appreciated that for certain agents, in particular peptide and polypeptide agents, it may be appropriate to select the peptide or polypeptide of the mammal, or a closely related molecule for administration. Thus, for example, it is preferred to administer human peptides or polypeptides to a human female. Of course, the peptide or polypeptide may be produced synthetically or by recombinant DNA means but would still be considered “mammalian” or, as the case may be “human”. Suitable IL-8 molecules and derivatives thereof are described in U.S. Pat. No. 5,624,670, incorporated herein by reference. Suitable MCP-1 molecules and derivatives thereof are described in U.S. Pat. No. 5,908,829, incorporated herein by reference.

It will be appreciated that the invention includes a method of ripening the female cervix, the method comprising administering a cervical ripening agent to the cervix using a needleless injector. The method can be used for the induction of labour at term (ie time of ordinary birth), but it may also be used for the induction of pre-term labour, and induction of labour in connection with a pathological pregnancy, or in connection with intrauterine fetal death. It may be used to ripen dysfunctional cervices, ie when dilation stops before completion. The method may also be used for preliminary cervical ripening prior to induction of abortion (eg in the first or second trimester abortion), and for induction of cervical ripening of a non-pregnant or pregnant female to assist surgical or diagnostic procedure such as D&C. Cervical ripening may also be induced in the female for the purposes of treatment by in vitro fertilisation.

It is particularly preferred if the cervical ripening agent is any one or more of IL-8, MCP-1 or a prostaglandin. The prostaglandin typically is PGE₂ which is currently used in cervical ripening. It is believed that by using the methods of the invention, which may be used to more accurately deliver the agent to a particular site on the cervix, a smaller amount of agent may be used compared to the use of gels, creams, pessaries and the like. At least in the case of PGE₂ this has the potential benefit of reducing the risk of undesirable contraction of the myometrium. The amount of PGE₂ may be reduced to 10% or 5% or 2% of the amount normally used when in a gel. For example, 2 to 100 μg of PGE₂ dissolved in 200 μl of sterile saline may be used.

The cervical ripening agent may be a combination of agents. It is particularly preferred if the cervical ripening agent is a complex comprising a proteinaceous agent which is chemotactic for monocytes and/or neutrophils and a prostaglandin.

By “proteinaceous agent which is chemotactic for monocytes and/or neutrophils” we include any protein or polypeptide or oligopeptide or peptide which is able to encourage monocytes and/or neutrophils into a tissue where the agent is present.

Typically, the agent is a proteinaceous cervical ripening agent.

By “proteinaceous cervical ripening agent” we include any protein or polypeptide or oligopeptide or peptide which has the ability to induce ripening of the cervix when suitably administered to the female. Thus, the proteinaceous cervical ripening agent is one which contains peptide bonds and has amino acid side chain residues.

Preferably, the proteinaceous agent which is chemotactic for monocytes and/or neutrophils is one with a pI value of 7.5 or above, preferably 8 or above and more preferably 8.5 or above. Typically, >5% of the amino acid residues are lysines.

The prostaglandin may be any suitable prostaglandin. It is preferred that the prostaglandin is one which is able to induce cervical ripening when suitably administered to the female. It is preferred that the prostaglandin is PGE₂ or a synthetic analogue thereof with a free acid. Synthetic analogues include these modified at position 15 by the addition of a methyl group.

It is particularly preferred if the prostaglandin is one which has an acidic group such as a carboxylate group. Preferably, the acidic group is one which, under low pH conditions, gives a free acid.

It is preferred that the prostaglandin is prostaglandin E₂ (PGE₂).

Particularly preferred proteinaceous cervical ripening agents include monocyte chemotactic peptide-1 (MCP-1) or interleukin-8 (IL-8) or variants thereof which retain the ability to induce cervical ripening.

These complexes are described in our co-pending PCT Patent Application which claims priority from UK Patent Application No 0103354.7.

A second aspect of the invention provides a system for delivering a biologically active agent to the cervix comprising an agent which is biologically active on the cervix and a needleless injector.

The needleless injector may be any needleless injector suitable for delivering the agent to the cervix. Such needleless injectors are described above and preferred injectors in the above mentioned methods of the invention are also preferred in this aspect of the invention.

The biologically active agent is an agent which is active on the cervix. Suitably, the agents are ones which are delivered to the cervix for therapeutic or prophylactic or other medical use. Typically, they are agents which are not ones which are administered to the skin. Suitable such agents include PGEs, IL-8 and MCP-1 as described above.

The agents preferably include cervical ripening agents as described above.

The system may be considered to be a kit of parts comprising various components. Thus, the kit of parts may comprise a needleless injector and, separately, the biologically active agent. In that case, the agent may be loaded into a suitable reservoir in the needleless injector. Alternatively, the kit of parts may comprise a needleless injector and a vial (by which we include capsule or cartridge or the like) which contains the agent wherein the vial is one which can be loaded into the injector. Additionally, in the case of powder injectors, the kit may contain the therapeutic agent in a suitable, powdered form. Similarly, in the case of liquid injectors, the kit may contain the therapeutic agent in a suitable solution.

It will be appreciated also that the system of this aspect of the invention may also comprise a needleless injector which is pre-loaded with the biologically active agent.

Vials (or capsules or cartridges) for use with a needleless injector are described in more detail below.

A third aspect of the invention provides a needleless injector loaded for injection with an agent which is biologically active on the cervix. The needleless injector may be any suitable needleless injector as described above, and the agent which is biologically active on the cervix may be any agent as described above. The needleless injector loaded with the agent according to this aspect of the invention may be formed by introducing a vial which contains the agent into the injector. Alternatively, the agent may be introduced into an existing reservoir within the needleless injector.

A fourth aspect of the invention provides a vial for insertion into, and containing an agent for delivery by, a needleless injector wherein the agent is an agent which is biologically active on the cervix.

The term “vial” may be interchanged with “cartridge”, “capsule” and the like, unless the context indicates otherwise. In particular, it includes the burstable membranes described in WO 94/24263 and the soft-walled capsules described in WO 96/12513.

Certain needleless injectors, for example those described in WO 93/03779, WO 95/03844, WO 00/10630, WO 94/24263, WO 96/20022 and WO 96/25190 are designed to receive pre-charged vials containing the agent to be “injected”. Thus, the vials of this aspect of the invention include such vials pre-charged with an agent which is biologically active on the cervix, such as those described above and, in particular, cervical ripening agents. Accordingly, the vials are of the correct size and shape to fit into the appropriate needleless injector. Also, the vials are made of appropriate material for their purpose. Suitably, the vial contains a unit dosage of the biologically active agent. Suitably, when used in a liquid needleless injector, the vial contains around 200 μl (for example, between 50 μl and 500 μl). Typical unit dosages are 2 to 100 μg of the agent, such as PGE2 or MCP-1 or IL-8.

Suitable vials for use with certain needleless liquid injectors, and methods of manufacture and filling the same, are described in WO 00/15281, WO 97/36785, WO 97/22375, WO 96/19252, WO 96/15821, WO 98/12121 and WO 98/13086, all of which are incorporated herein by reference.

Suitable vials for use with certain needleless powder injectors, and methods of manufacturing and filling the same, are described in WO 00/54827, WO 00/62846, U.S. Pat. No. 5,780,100, WO 98/21364, WO 99/01169, WO 96/20022, WO 96/12513 and WO 94/24263.

A fifth aspect of the invention provides a method of preparing a needleless injector for use in delivering a biologically active agent to the cervix, the method comprising loading the injector with the biologically active agent. The biologically active agent may be loaded directly into a pre-existing eservoir within the needleless injector or it may be loaded into the injector in a pre-charged vial.

A sixth aspect of the invention provides a pharmaceutical formulation comprising an agent for delivery to the cervix and a carrier suitable for use in a needleless injector.

The pharmaceutical formulation is typically sterile and pyrogen free.

For liquid injectors the biologically active agent is typically dissolved in sterile water or sterile saline. However, it may be advantageous to include a dispersion agent or an agent which allows for or increases transdermal or transmucosal transfer or penetration such as dimethyl sulphoxide (DMSO) and the like.

For powder injectors the powder to be delivered needs to be of a suitable quality for delivery. Suitable powder (ie particle compositions) may be made according to the methods described in WO 97/48485 and WO 00/53160, incorporated herein by reference. In particular, the particles (powder) for use in the present invention have an optimum particle density ranging from about 0.1 to about 25 g/cm³, preferably ranging from about 0.5 to about 3.0 g/cm³, and most preferably ranging from about 0.8 to 1.5 g/cm³. The densified particles are suitably processed to obtain particle sizes ranging from about 0.1 to 250 μm, preferably ranging from about 0.1 to 150 μm, and most preferably ranging from about 20 to about 60 μm.

Thus, the invention includes particles comprising a cervical ripening agent such as interleukin-8 or MCP-1 or PGE₂ or combinations thereof with these physical parameters. These particles may be made using the methods described in WO 97/48485, incorporated herein by reference. The particles may be biologically inert particles onto which has been coated the biologically active agent. Suitably, the biologically active agent may be deposited onto the particle by dissolving it in a suitable solvent and then evaporating the solvent.

A further aspect of the invention provides use of a cervical ripening agent in the manufacture of a medicament for treating a female in need of a cervical ripening agent wherein the cervical ripening agent is for delivery using a needleless injector.

Preferably, the cervical ripening agent is any one or more of IL-8, MCP-1 or prostaglandin E₂.

The invention will now be described in more detail by reference to the following Examples and Figures.

FIG. 1: Anatomy of the human vagina, cervix and uterus.

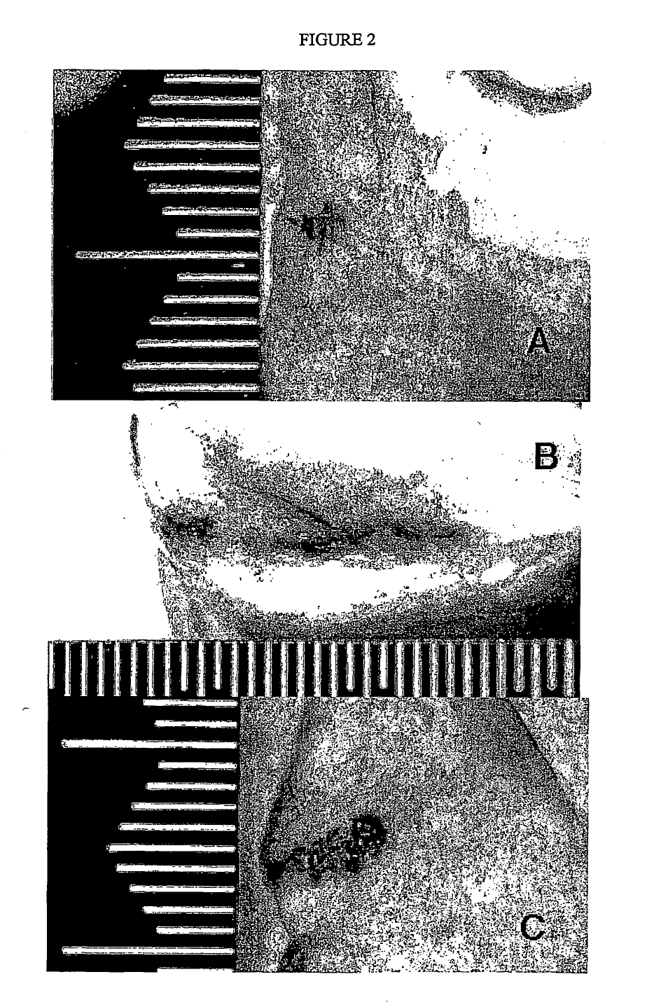

FIG. 2: View of dye track for three separate cervices injected with the Weston needleless injector. Note the small ruler divisions are in mm. All injections were performed from the face of the cervix that would normally be in the vagina.

### EXAMPLE 1

### Administration of MCP-1 to a Human Female Using a Needleless Syringe

The patient is a human female in need of cervical ripening.

200 μl of solution of monocyte chemotactic peptide-1 (MCP-1) of 100 μg/ml in sterile saline is loaded into a helium-loaded injection device and the end of the device is placed either in the vaginal fornix against the cervical mass or against the exposed cervical lumen. The injector is fired at one or two locations.

### EXAMPLE 2

### Preparation of IL-8 as a Powder for Injection Using a Needleless Injector

Lyophilized recombinant human interleukin-8 powder is obtained and reprocessed using the method described in WO 97/48485. Particularly, approximately 30 mg of IL-8 was compacted under pressure using a Carver Laboratory Pellet Press (Model 3620, available from Carver, Inc., Wabash, Ind.). The pressure of compaction was 15,000 lbs/in², which was applied for approximately 45 seconds. A pellet was obtained which was ground using mortar and pestle until visually broken up. The resulting reduced pellet was then sieved using a 53 μm sieve (Endecott, London). Particles having a size greater 53 μm were selected and appropriate dosages thereof were measured into drug cassettes for delivery from a needleless syringe.

### EXAMPLE 3

### Use of the Weston Needleless Injector to Evaluate Penetration of the Cervix

A Weston needleless injector was loaded with 0.2 ml of dye (1 mg/ml of methylene blue {Sigma} in phosphate buffered saline). Whole human cervix was obtained at hysterectomy from non-pregnant women of reproductive age. The cervix was tested within 30 minutes of removal from the patient. The needleless injector device was held against the ectocervix region. Increased pressure on the needleless injector activated the device. Two injections were performed for each specimen.

The cervix was dissected along the track of the injection (FIG. 2) and the length of the dye track was measured. In four experiments the length of the track was a mean of 6.1 mm (standard error of the mean=1.9 mm). The mean lateral spread (any signs of the blue dye) was 8.8 mm.

Liquid needleless injectors (eg Weston) penetrate skin but the liquid stream rebounds when it hits muscle. The cervix has relatively little smooth muscle (but gets its strength from collagen) and therefore the extent to which the liquid stream would penetrate was unknown until these experiments. As far as we can see there is relatively little tissue damage at the site of injection. The lateral spread of the injected dye is very difficult to see in the black/white photographs but can be seen in the colour ones from which the black and white photographs are derived.

The use of the needleless injector in testing the penetration and spread of a marker dye shows that the distribution of cervical ripening product would be ideal for a rapid ripening of the cervix. The relatively wide spread of the injected material suggests that the ripening agent would get ready access to the vasculature of the cervix. The rapid distribution will reduce the time for any agent to start the ripening process. Thus the use of such a device together with an effective cervical softening agent will provide the obstetrician with a useful tool to control the cervical ripening process.

1. A method of delivering a biologically active agent to the cervix, the method comprising using a needleless injector.

2. A method according to claim 1 wherein the needleless injector is a liquid injector.

3. A method according to claim 1 wherein the needless injector is a powder injector.

4. A method according to claim 1 wherein the biologically active agent is a cervical ripening agent.

5. A method according to claim 4 wherein the cervical ripening agent is any one or more of a prostaglandin, MCP-1 and IL-8.

6. A method according to claim 1 wherein the biologically active agent is a vasodilator or precursor thereof, a chemokine or a cytokine which stimulates monocyte or granulocyte entry into cervical tissue.

7. A method of ripening the female cervix, the method comprising administering a cervical ripening agent to the cervix using a needleless injector.

8. A system for delivering a biologically active agent to the cervix comprising an agent which is biologically active on the cervix and a needleless injector.

9. A system according to claim 8 wherein the biologically active agent is a cervical ripening agent.

10. A system according to claim 8 wherein the needleless injector is a liquid injector.

11. A system according to claim 8 wherein the needleless injector is a powder injector.

12. A needleless injector loaded for injection with an agent which is biologically active on the cervix.

13. A needleless injector according to claim 12 wherein the biologically active agent is a cervical ripening agent.

14. A needleless injector according to claim 12 which is a liquid injector.

15. A needleless injector according to claim 12 which is a powder injector.

16. A vial for insertion into, and containing an agent for delivery by, a needleless injector wherein the agent is an agent which is biologically active on the cervix.

17. A vial according to claim 16 wherein the agent is a cervical ripening agent.

18. A method of preparing a needleless injector for use in delivering a biologically active agent to the cervix, the method comprising loading the injector with the biologically active agent.

19. A method according to claim 18 wherein the agent is loaded in a vial disposed for insertion into the needleless injector.

20. A pharmaceutical formulation comprising an agent for delivery to the cervix and a carrier suitable for use in a needleless injector.

21. A pharmaceutical formulation according to claim 20 wherein the formulation contains particles of a density between about 0.1 and about 25 g/cm³ and of a size between 0.1 and 250 μm which particles comprise the said agent.

22. A pharmaceutical formulation according to claim 20 comprising an agent which permeabilises a mucosal surface, such as dimethylsulphoxide.

23. Use of a cervical ripening agent in the manufacture of a medicament for treating a female in need of a cervical ripening agent wherein the cervical ripening agent is for delivery using a needleless injector.

24-26. (canceled).
